(19) United States
(12) Patent Application Publication
(10) Pub. No.: US 2024/0157361 A1
(43) Pub. Date: May 16, 2024

(54) MICROFLUIDIC DEVICE

(71) Applicant: PHARMAFLUIDICS, Gent (BE)

(72) Inventors: Gert Desmet, Zemst (BE); Paul Jacobs, Lokeren (BE); Jeff OP DE BEECK, Mariakerke (BE); Bo CLAEREBOUT, Kortrijk (BE)

(21) Appl. No.: 18/550,253

(22) PCT Filed: Mar. 11, 2022
(86) PCT No.: PCT/IB2022/052209

§ 371 Date: Sep. 12, 2023

(30) Foreign Application Priority Data
Mar. 12, 2021 (BE) 2021/5188

(51) Int. Cl. B01L 3/00

(52) U.S. Cl. CPC B01L 3/502761; B01L 3/502707; B01L 2200/0647; B01L 2400/086

(57) ABSTRACT

A microfluidic device (100) comprising: a substrate (110) having a liquid channel (120), an ordered set of pillars (130) positioned in the channel (120), the individual pillars (130) comprising at least one pair of fins that form a chevron-shaped cross-section with the substrate, and being arranged in pairs of rows, adjacent rows being laterally displaced with respect to one another by half a pillar in length, the pillar length being measured perpendicular to the average liquid direction, thereby forming microchannels between the pillars, and the rows being staggered so that the microchannels formed between pillars of successive rows at each position along the longest pillar side have substantially the same width.

### SCOPE OF THE INVENTION

This invention relates in general to microfluidic devices for chemical reactors. More specifically, the invention relates to the pillar bed of a microfluidic device.

### BACKGROUND OF THE INVENTION

Microfluidic devices that use liquid propagation within them have a large number of applications. Examples include the production of chemical components, the synthesis of nanoparticles, the separation and/or extraction of components, etc.

A specific example of a separation technique for separating mixtures, e.g. to be able to analyse them accurately, is chromatography. Various forms of chromatography exist, such as gas chromatography, gel chromatography, thin-layer chromatography, adsorption chromatography, affinity chromatography, liquid chromatography, etc.

Liquid chromatography is typically used in the fields of pharmacy and chemistry for both analytical and production applications. Liquid chromatography uses the difference in affinity of different substances with a mobile phase and a stationary phase. As each substance has its own “adhesion force” up to the stationary phase, they are carried along faster or slower with the mobile phase, meaning that certain substances can be separated from others. It is essentially applicable to any compound, has the advantage that no evaporation of the material is required, and has the advantage that variations in temperature have only a negligible effect.

An efficient form of liquid chromatography is high-pressure liquid chromatography (also known as high-performance liquid chromatography), HPLC, in which high pressure is applied to the separation process. A specific example of a technique to perform HPLC is based on chromatographic columns via pillars. Since their introduction to liquid chromatography, pillar-based chromatographic columns have proven to be a worthy alternative to systems based on packed-bed structures and monolithic systems. Due to the ability to apply the columns with a high degree of uniformity and order them perfectly, the dispersion resulting from differences in current paths or “eddy dispersion” can be prevented almost completely. This principle is more generally capable of application in chemical reactors based on liquid plug propagation.

It is also known from the theory of chromatography that, in addition to the uniformity of the separation bed, it is important that the distances covered by the molecules due to diffusion should be as small as possible. Translated to a pillar bed, this design requires narrow pillars that are arranged in close proximity. Furthermore, flow simulations also show that the zones between the pillars, the microchannels, should ideally have as uniform a width as possible.

To enable sufficiently high liquid flow rates, the pillar beds must be sufficiently deep (since the width of the pillar beds is limited by the usable surface area of the silicon wafers). In combination with the demand for narrow columns, this results in columns with a high aspect ratio. However, this increases the risk that the pillars will collapse (stiction) during the various wet treatment processes required during production. Consequently, there is scope for improvement in microfluidic devices that are built up from a channel with an ordered set of pillars in it.

### SUMMARY OF THE INVENTION

One objective of the embodiments of the present invention is to produce a good microfluidic device with pillars.

It is an advantage of the embodiments produced according to the present invention that the pillars produced permit a good aspect ratio and good pillar density, and at the same time reduce the risk of pillars collapsing/compacting during the production process.

The aforementioned object is achieved by an apparatus, device and/or method that corresponds to the present invention.

In a first aspect, the present invention relates to a microfluidic device based on a liquid flow. The microfluidic device contains:
A substrate having a liquid channel defined by channel walls, the channel having an inlet and an outlet, and the channel having a longitudinal axis in accordance with the average liquid flow direction of a liquid as it flows in the channel from the inlet to the outlet;
An ordered set of pillars positioned in the channel on the substrate, the individual pillars comprising at least one pair of fins, the fins forming a chevron-shaped cross-section with the substrate.

The pillars are arranged in pairs of rows. Steam openings are located between the pillars of the same row, these also being called nodes. The rows are arranged in a staggered manner with respect to one another and are parallel to one another, so that microchannels between the pillars of two successive rows have substantially the same width. In addition, adjacent rows are laterally displaced with respect to one another over half a pillar length, the pillar length being measured perpendicular to the average liquid direction and parallel to the substrate. The nodes of adjacent rows are therefore also displaced with respect to one by half a pillar length. One advantage of the embodiments of the present invention is that the pillars can be placed closer to one another on average, and that uniform flow pores can be achieved that are narrower and/or higher than with existing pillars. For the present invention, this is achieved by using columns with a chevron-shaped cross-section.

In embodiments of the present invention, the flow pores (the microchannels between the pillars) are preferably of equal width throughout.

In embodiments of the present invention, the chevron form is such that a substantially constant microchannel width is obtained between two adjacent pillars of the same row.

In embodiments of the present invention, the ratio of the total width Bₜ of a pillar, measured in the average liquid flow direction, and the average width Bᵢ of the pillar, measured perpendicular to a wall of a fin, is greater than 1.05.

One advantage of embodiments of the present invention is that the structural rigidity is increased compared to, for example, a pillar shape with a rectangular cross-section with the same width Bᵢ, length and height. This allows structures with a larger aspect ratio to be obtained.

In embodiments of the present invention, the pillars that touch the channel walls contain only half the fins of a pillar that does not touch the channel wall.

In embodiments of the present invention, the outer pillars touch the channel walls in one row of a pair of rows, and there are flow openings between the outer pillars and the channel walls for the other row of the pair. In embodiments of the present invention, the fins that touch the channel walls are arranged in the same direction. This means that the angle formed between the channel wall and the fin, for example, measured on the side which reaches the liquid flow first, is the same for the fins on both channel walls.

In embodiments of the present invention, for one row of a pair of rows there is a flow opening between an outermost pillar and a first channel wall, with a pillar on the other side touching a second channel wall opposite the first channel wall, and for the other row of the pair of rows there is a flow opening between an outermost pillar and the second channel wall, with a pillar on the other side touching the first channel wall.

As already discussed above, in embodiments of the present invention flow openings are located between adjacent pillars of the same row. These are also called double nodes.

There may also be an opening between the channel wall and a pillar. This is called a single node.

Examples in which there is one row per pair of rows, with a single node on both sides, and a row where there is no opening between the end pillars and the channel walls, can be seen in FIG. 2 to FIG. 6.

An example in which in each row there is one and only one single node per row present on one channel wall and none on the other channel wall, and in which the single node in one row of the pair of rows is located on a particular channel wall of the channel, and the single node in the other row of the pair of rows is located on the other channel wall, is illustrated in FIG. 7.

In embodiments of the present invention, the connections between two fins and the ends of the pillars are rounded.

In embodiments of the present invention, the ratio of the height of the pillars and the width of the pillars is greater than three. The height of the pillars is measured in a direction orthogonal to the substrate.

In embodiments of the present invention, the fins of the pillars have a width (Bₚ) in the direction of the longitudinal axis of the channel, and the chevron shapes have a length (L_{c}) in a direction perpendicular to the longitudinal axis and parallel to the substrate, with the individual chevron shapes having a length-width ratio of at least three.

In embodiments of the present invention, the ends of the fins are parallel to the channel walls.

In embodiments of the present invention, the microfluidic device contains a top plate on top of the set of pillars, the top plate being positioned opposite the substrate.

In embodiments of the present invention, the pillars are microfabricated pillars.

In embodiments of the present invention, the smallest distance (W₀) between two adjacent pillars is between 0.5 times and 0.8 times the smallest distance (d1) between the channel wall and an adjacent, non-contacting pillar.

In embodiments of the present invention, the microfluidic device is a liquid chromatography separation device.

In a second aspect, the present invention relates to a mask for lithographically applying a structure in a substrate for the manufacture of a microfluidic device. The mask comprises design elements for defining an ordered set of pillars positioned in the channel on the substrate, the individual pillars having at least one pair of fins that form a chevron-shaped cross-section with the substrate.

The pillars are arranged in pairs of rows. The rows are arranged in a staggered manner with respect to one another and are parallel to one another, so that microchannels between the pillars of two successive rows have substantially the same width. In addition, juxtaposed rows are laterally displaced with respect to one another over half a pillar length, the pillar length being measured perpendicular to the average liquid direction and parallel to the substrate.

In a third aspect, the present invention relates to a method for producing a microfluidic device, the method comprising the lithographic implementation of a channel with pillars using a mask in accordance with the present invention.

Specific and preferred aspects of the invention are included in the attached independent and dependent claims. Characteristics of the dependent claims can be combined with characteristics of the independent claims and with characteristics of other dependent claims, as indicated, and not only as expressly stated in the claims.

These and other aspects of the invention will be clear from and clarified with reference to the embodiment(s) described below.

### BRIEF DESCRIPTION OF THE FIGURES

FIG. 1 shows a top view of a chromatographic column with pillars according to the previous art.

FIG. 2 shows a top view of a microfluidic device according to embodiments of the present invention with one chevron shape per pillar.

FIG. 3 shows a top view of a microfluidic device according to embodiments of the present invention with two chevron shapes per pillar.

FIG. 4 shows a top view of a microfluidic device according to embodiments of the present invention with four chevron shapes per pillar.

FIG. 5 shows a top view of a microfluidic device according to embodiments of the present invention with pillars with rounded corners.

FIG. 6 shows a top view of a microfluidic device according to embodiments of the present invention with undulating pillars.

FIG. 7 shows a top view of a microfluidic device according to embodiments of the present invention with single nodes adjoining an alternating side wall.

FIG. 8 shows a chevron structure with a shaded portion that, when part of the chevron structure, provides a more uniform flow width at the nodes, in accordance with embodiments of the present invention.

FIG. 9 illustrates steps for producing a microfluidic device in accordance with embodiments of the present invention.

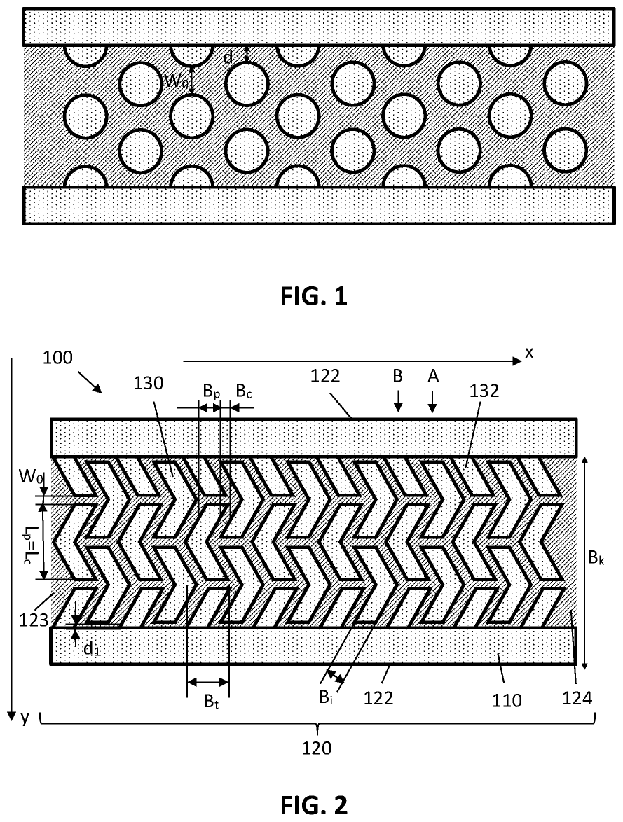

The figures are for schematic purposes only and are not limiting. In the figures, the dimensions of some parts may be exaggerated and not presented to scale for illustrative purposes.

Reference numbers in the claims should not be interpreted as limiting the scope of protection. In the various figures, the same reference numbers refer to the same or similar elements.

### DETAILED DESCRIPTION OF ILLUSTRATIVE EMBODIMENTS

The present invention is described with reference to particular embodiments and with reference to certain drawings, though the invention is not limited thereto; it is limited exclusively by the claims. The described drawings are for schematic purposes only and are not limiting. In the drawings, the dimensions of some elements may be exaggerated and not drawn to scale for illustrative purposes. The dimensions and relative dimensions do not always correspond to the actual practical embodiment of the invention.

Furthermore, the terms “first”, “second”, “third” and the like in the description and in the claims are used for distinguishing similar elements and not necessarily for describing an order, whether in time, space, order of precedence or any other way. It should be understood that the terms used in this way are interchangeable under suitable circumstances, and that the embodiments of the invention described herein are suitable for operation in an order that differs to the one described or represented herein.

Moreover, the terms “top”, “bottom”, “above”, “before” and the like in the description and the claims are used for descriptive purposes and not necessarily for describing relative positions. It should be understood that the terms used in this way can be interchanged under certain circumstances, and that the embodiments of the invention described herein are also suitable for operation according to orientations other than those described or represented herein.

It should be noted that the term “contains”, as used in the claims, should not be interpreted as limited to the means described thereafter; this term does not exclude other elements or steps. It can therefore be interpreted as specifying the presence of the features, values, steps or components referred thereto, but does not exclude the presence or addition of one or more other features, values, steps or components, or groups thereof. Therefore, the scope of the expression “a device containing the means A and B” should not be limited to devices consisting exclusively of components A and B. With regard to the present invention, it means that A and B are the only relevant components of the device.

Reference throughout this specification to “one embodiment” or “an embodiment” means that a specific feature, structure or characteristic described in connection with the embodiment is included in at least one embodiment of the present invention. Therefore, the occurrence of the expressions “in one embodiment” or “in an embodiment” at various places throughout this specification need not necessarily always refer to the same embodiment, but may do so. Furthermore, the specific features, structures or characteristics can be combined in any suitable manner, as would be clear to an average person skilled in the art on the basis of this publication, in one or more embodiments.

Similarly, it should be appreciated that, in the description of illustrative embodiments of the invention, several features of the invention are sometimes grouped together in a single embodiment, figure or description thereof, with the aim of streamlining the publication and helping to understand one or more of the various inventive aspects. In any event, this method of publication should not be interpreted as reflecting the intention that the invention requires more features than are explicitly mentioned in each claim. Rather, as the following claims reflect, inventive aspects are present in less than all of the features of a single prior disclosed embodiment. Therefore, the claims following the detailed description are hereby explicitly included in this detailed description, with each independent claim constituting a separate embodiment of this invention.

Furthermore, while some embodiments described herein contain some, but not other features included in other embodiments, combinations of features of various embodiments are intended as being within the scope of the invention, and these form various embodiments as would be understood by those skilled in the art. For example, in the following claims any of the described embodiments may be used in any combination.

Numerous specific details are given in the description provided here. In any case, it can be understood that embodiments of the invention can be produced without these specific details. In other cases, well-known methods, structures and techniques have not been shown in detail to ensure the clarity of this description.

Where reference is made in the present description and claims to microchannels between the pillars, this infers channels in which at least one of the dimensions lies in the interval of 10 μm to 0.1 μm.

Where reference is made in the current description and claims to an ordered set, this infers a set of elements that is not positioned randomly, but where there is a specific relationship between the distances of the elements to one another.

Where reference is made in the present description and claims to distribution or dispersion, this infers the spatial distribution over an area or volume.

Where reference is made in the present description and claims to the permeability, this infers the flow rate at which a liquid can flow through the liquid channel with pillars for a given pressure gradient and channel length.

Where reference is made in the present description and claims to the aspect ratio of a pillar, this infers the ratio between the height and the smallest dimension of the pillar. In embodiments of the present invention, this smallest dimension is the width of the pillar, the width being measured in the average liquid flow direction.

Where reference is made in the present description and claims to the form factor of a pillar, this infers the length/width ratio, that is to say the length of the pillar being measured at right angles to the average liquid direction.

In the first aspect, the present invention relates to a microfluidic device 100 based on a liquid flow. The microfluidic device is typically suitable for the propagation of a liquid plug. In accordance with embodiments of the present invention, the microfluidic device can be a liquid chromatography device, although embodiments are not limited hereto. Another specific example is a gas chromatography device. The microfluidic device may be more generally suitable for producing certain components, such as intermediates, for synthesising components such as nanoparticles, for separating and/or extracting components, etc.

Examples of a microfluidic device in accordance with embodiments of the present invention are shown in FIG. 2 to FIG. 6. The same reference numbers are used for each part throughout the various figures.

According to embodiments of the present invention, the microfluidic device 100 contains a substrate 110 with a liquid channel 120, also known as a fluid channel. The channel 120 has an inlet 123 and an outlet 124 for the supply and discharge of the fluid. A longitudinal axis of the channel 120 is in accordance with the average liquid flow direction of a liquid as it flows in the channel 120 from the inlet 123 to the outlet 124.

In embodiments of the present invention, the microfluidic device comprises an ordered set of pillars 130 positioned in the channel 120 on the substrate 110, the individual pillars 130 comprising at least one pair of fins, the fins forming a chevron-shaped cross-section with the substrate. In a chevron-shaped pillar, a pair of fins is arranged at an angle to one another.

In embodiments of the present invention, the pillars 130 are arranged in pairs of rows (see, for instance, the examples in FIG. 2 to FIG. 6). Adjacent rows are arranged in a laterally displaced manner with respect to one another. The displacement amounts to half a pillar in length. The pillars are arranged in such a way that microchannels are formed between the pillars, so that these microchannels between pillars of successive rows have substantially the same width B_{c} (for example, with a deviation of less than 10%, or even less than 5%, or even less than 1%, or even less than 0.1%, and in a preferred embodiment of even 0%).

While in FIG. 2 the liquid flows from left to right, the concave side of the chevron pairs of row A then faces the average liquid flow, and the convex side of the chevron pairs of row B faces the average liquid flow.

In embodiments of the present invention, the pillars are arranged in such a way and the chevron form is such that a substantially constant microchannel width is obtained between two adjacent pillars of the same row.

One advantage of embodiments of the present invention is that a greater aspect ratio of the pillars can be obtained compared to pillars with a circular or other regular cross-section. The chevron-shaped cross-section reduces the risk of collapsing pillars.

In addition, it is an advantage of the embodiments of the present invention that, for a given minimum inter-pillar distance, a higher pillar density can be obtained compared to an ordered structure of pillars with a circular cross-section. In embodiments of the present invention, the pillars have a greater form factor than, for example, a pillar with a circular, square, triangular, or equilateral polygon cross-section. In embodiments of the present invention, the structures can be made larger in an isomorphous sense up to the point where the entire flow pore (i.e. the microchannel between pillars of adjacent rows) is given zero width throughout (except in the sections directed in the main flow direction).

In addition, it is an advantage of the embodiments of the present invention that the average distance between the pillars is always as close as possible to the minimum distance between the pillars, so that flow paths with as uniform a width as possible are obtained.

In embodiments of the present invention, all segments of flow paths are given equal flow rates to process. To achieve this, the pillars are arranged symmetrically with respect to these nodes, where the naming expresses symmetrically that pillars are arranged in such a way that in each node where two branches arrive and where two branches depart, the flow rate in each of the four branches is substantially the same. Such a node, where two branches arrive and where two branches depart, is called a double node.

It is an advantage of the embodiments of the present invention that pillars with a sufficiently high form factor can be achieved, thereby reducing wall effects on the liquid flow.

In addition, it is an advantage of the embodiments of the present invention that the distribution of the sample plug as it migrates through the column can be reduced by introducing rows of chevron-shaped pillars ordered in such a way that a substantially constant microchannel width is obtained between adjacent pillars of the same row. The rows are laterally displaced with respect to one another by half a pillar length. In embodiments of the present invention, the chevron-shaped pillars are ordered in such a way that a substantially constant microchannel width is obtained between the pillars. The rows are staggered with respect to one another, so that the convex part of one row is aligned with the concave part of another row and vice versa, so that microchannels of equal width are present between the rows. The openings (nodes) in the rows are arranged in such a way that the microchannels of those arriving in a double node are symmetrical, and that the microchannels departing in a double node are symmetrical. The symmetry axis is a longitudinal axis through the double node. A microchannel enters a node when a liquid flow is present in the microchannel in the direction of the node during operation of the microfluidic device. A microchannel departs from a node when a liquid flow is present in the microchannel away from the node during operation of the microfluidic device. It is an advantage of such symmetrical double nodes that the uniformity of the flow field can be increased compared to nodes that do not have this symmetry.

In embodiments of the present invention, the pillars (except those on the side, where they are half the length) have the same form and are of the same size. In some embodiments of the present invention, any angle between the fins of an individual pillar is substantially the same for all pillars.

The substrate can be any suitable substrate, such as a polymer substrate, semiconductor substrate, metal substrate, ceramic substrate or glass or vitreous substrate. For example, the substrate may be a typical microfluidic substrate. The fluid channel may be a channel formed in or on the substrate.

In embodiments according to the present invention, the microfluidic device contains a top plate on top of the set of pillars, the top plate being positioned opposite the substrate 110. This top plate is in contact with the pillars. In a specific embodiment, the invention not being limited to this, the fluid channel is introduced as a depression in the substrate and a second substrate (the top plate) is introduced on top of the first substrate so as to obtain a fluid channel which is closed at the top, side and bottom. The channel may have a rectangular cross-section. In embodiments of the present invention, the pillars extend from the substrate to the top plate. In embodiments of the present invention, the pillars extend from the substrate to the top plate to obtain a flow field that is as uniform as possible.

In an embodiment of the present invention, the channel being introduced as a depression in a first substrate and being covered by a second substrate, the inlet and the outlet can have perforations in the first and/or second substrate.

The fluid channel may have a length that depends on the application. By using specific inlet structures and/or outlet structures, for example distributors, it is also possible to influence the required length. A typical width of the liquid channel can be chosen as needed. The necessary width will typically depend on the chosen length and vice versa. In a set of examples, the width of the fluid channel Bₖ can be chosen at an interval of 0.02 mm to 250 mm.

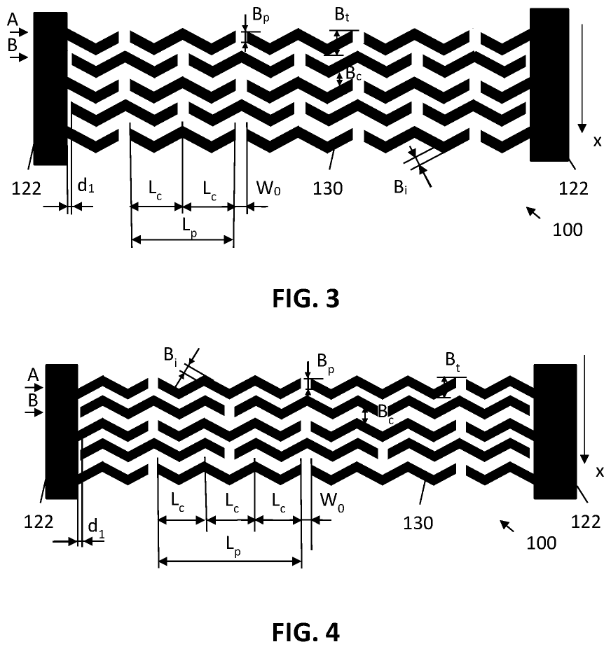

For the liquid channel 120, a longitudinal axis can typically be defined, the longitudinal axis being located according to the direction of the average flow direction of the fluid in the channel from inlet to outlet. By way of illustration, the longitudinal axis in the schematic example of the microfluidic device 100 shown in FIG. 2 is as the x-axis. This x-axis is also shown in FIG. 3 to FIG. 5. Furthermore, the substrate 110, the channel 120 itself and the channel wall 122 are also indicated in FIG. 2. The channel wall 122 defines the liquid channel 120. It should be noted that the channel wall 122 may be defined by the substrate material.

The pillars may be micro-manufactured pillars, although embodiments are not limited hereto. The pillars can be based on precision manufacturing techniques. According to embodiments of the present invention, the pillars 130 have a chevron-shaped cross-section with the substrate.

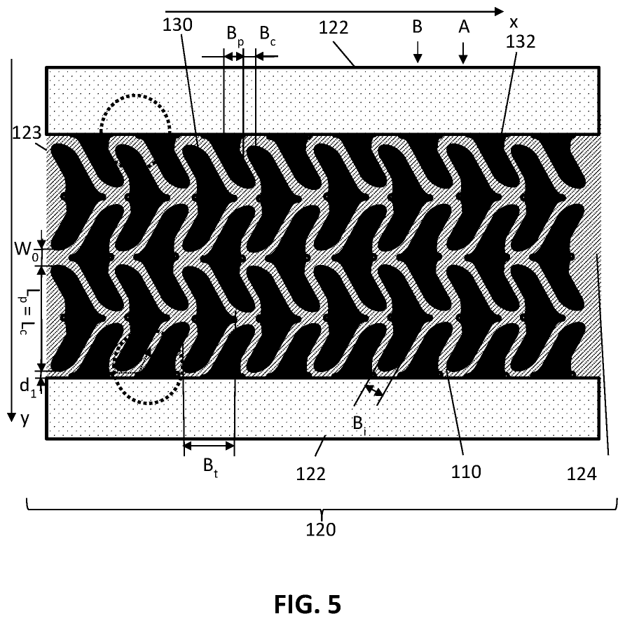

In some embodiments of the present invention, the pillars are of equal width throughout. In the examples illustrated in the figures, this width is denoted as Bₚ. Where reference is made to the width Bₚ in embodiments of the present invention, this refers to the width measured following projection of a fin section onto the x-axis. In FIGS. 2, 3 and 4, the pillars are of equal width throughout. In FIG. 5, the width Bₚ of the pillars varies. This width B corresponds to the average width of a pillar measured perpendicular to the wall. This can vary, for example, between 0.5 μm and 5 μm, or up to 10 μm, or up to 50 μm.

Where reference is made to the height of a pillar in embodiments of the present invention, this is measured perpendicular to the substrate. Where reference is made in embodiments of the present invention to the length L_{c} of a chevron shape, this refers to the distance measured in a direction parallel to the substrate and perpendicular to the longitudinal axis. The length of a pillar Lₚ is the total length of the pillar measured in the direction parallel to the substrate and perpendicular to the longitudinal axis.

In embodiments of the present invention, the ratio of the height of the pillars and the width Bₚ of the pillars is greater than 8 or even greater than 10. It is an advantage of the embodiments of the present invention that a greater height/width ratio can be obtained by using pillars with a chevron-shaped cross-section than by using cylindrical pillars. This ratio is also called the aspect ratio. To avoid stiction (the collapse of pillars), the chevron structures are introduced, which permit a higher aspect ratio.

In embodiments of the present invention, the ratio between the length L_{c} and the width Bₚ of the chevron structure is greater than 6, or greater than 7, or even greater than 8. This length-width ratio is also called the form factor. For example, the form factor may be between 6 and 9, but may also be, for example, as high as 20 or 30.

At the same time, the ends of the fins are shaped in such a way that the width W₀ of the channel between two fins of the same row is virtually constant (see, for example, FIGS. 2, 3 and 4). In some embodiments of the present invention (see, for example, FIG. 4), there may still be some variation in the width W₀ of the channel between two fins of the same row. The variation of the width may, for example, be less than 20% or even less than 10%. In embodiments of the present invention, the angle between the fins of a chevron structure may, for example, be between 5° and 175°.

In embodiments of the present invention, the ends of the fins are parallel to the channel walls. It is an advantage of the embodiments of the present invention that the microchannels formed between the ends of adjacent fins are parallel to the average liquid flow direction.

In embodiments of the present invention, the channels formed between two pillars of successive rows of are of a substantially equal width at the places where this width can be measured. In the figures, this width is denoted as B_{c} and is measured following projection of a channel section on the x-axis. This can vary, for example, between 0.1 and 10 μm.

In embodiments of the present invention, the total width Bₜ of the pillar in the axial direction is greater than x times the average width Bᵢ of the pillar. The width Bᵢ is measured in a direction perpendicular to the wall of the fin. The width can be measured at various places, and the average calculated. The ratio x may, for example, be greater than or equal to 1.05.

In embodiments of the present invention, the width W₀ in the y-direction (perpendicular to the x-direction and parallel to the substrate) of the flow opening in the nodes can be freely chosen. In preferred embodiments of the present invention, this width is less than or equal to 2*B_{c}. This width can be chosen, for example, in an interval between 0.5 μm and 5 μm. In embodiments of the present invention, as shown in FIG. 2 to 4, this width W₀ is virtually constant.

In embodiments of the present invention, a porosity=zero can be achieved in the special case where the flow opening W_{O}=B_{c}. As already described above, this is obtained with isomorphic pillars.

It is an advantage of the embodiments of the present invention that the smallest distance between two adjacent pillars can be chosen to be smaller than the smallest distance between the channel wall and an adjacent non-contacting pillar, without edge effects occurring. In some embodiments of the present invention, the smallest distance (W₀) between two adjacent pillars 130 is between 0.5 times and 2 times, or even between 0.5 times and 1.5 times, or even between 0.5 times and 1.1 times the smallest distance (d1) between the channel wall 122 and an adjacent, non-contacting pillar.

In an embodiment of the present invention, the microfluidic device is a liquid chromatography separation device. In these microfluidic devices, the liquid channel is a separate column. It is an advantage that the separation efficiency of the system can be high due to the large lateral migration, but that no marginal effects occur or that these effects are negligible.

The number of pillars introduced in the channel can be chosen according to the objective that must be achieved (the separation capacity, for example). The number of columns that can be produced in the liquid channel in a particular row depends on the width of the channel. For example, between 2 and 1,000 columns per mm width of the channel can be introduced.

In embodiments of the present invention, the individual pillars contain exactly one pair of fins. In other embodiments, a pillar may contain two pairs of fins. In FIG. 2, the number of chevron structures within a single pillar is equal to two. In FIG. 3, there are two chevron structures per pillar. In this example, there are four pillars in a single row. However, there may also be more or fewer.

There may also be more chevron structures per pillar. An example of this is shown in FIG. 4, where there are three chevron structures (six pairs of fins) per pillar. In this case, too, in embodiments of the present invention, there may be more pillars present in a single row.

In embodiments of the present invention, the pillars 132 that touch the channel walls 122 contain half the fins of a pillar that do not touch the wall (for example, only one fin of the chevron form where the pillar that does not touch the wall consists of two fins).

In some embodiments of the present invention, the fins that touch the channel walls are only present in the first row of the row pairs. In some embodiments of the present invention, by contrast, they are present only in the second row of the row pairs. In these cases, there are either two pillars touching the wall in the odd rows (and no pillars touching the wall in the even rows) or two pillars touching the wall in the even rows (and no pillars touching the wall in the odd rows).

In embodiments of the present invention, the fins that touch the channel walls are arranged in the same direction. This can be, for example, the direction of the average liquid direction.

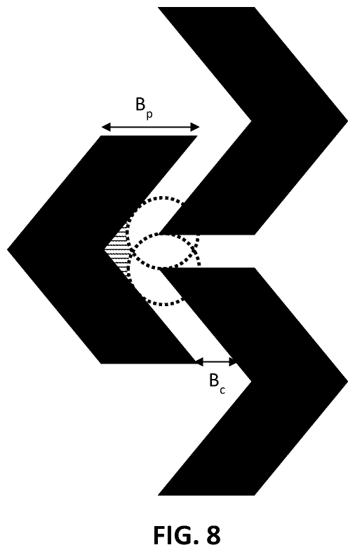

In embodiments of the present invention, the connections between two fins and the ends of the fins are rounded. An example of this can be seen in FIG. 5. The chevron structures in FIG. 5, similar to the examples in FIG. 2, 3 and FIG. 4, also comprise fins positioned at a certain angle to each other. The corners of the chevron structure are rounded in this example. In addition, protuberances are applied centrally, both on the concave side and on the convex side of the pillars. These protuberances ensure that a more uniform flow width is obtained. This is further illustrated in FIG. 8. The shaded area gives the pillar a shape that obtains a more uniform flow width at the nodes. This area can be seen as a transition to the chevron structures of FIG. 5.

As already discussed above, in embodiments of the present invention, the fins on the channel walls are oriented in the same direction as the flow direction. This means that the angle formed between the channel wall and the fin, measured on the side which first reaches the liquid flow, is greater than 90°. In embodiments, this angle may, for example, be between 91° and 179°, between 100° and 170° or between 100° and 140°. In embodiments of the present invention, the fins 132, in contact with the channel wall, are parallel to the adjacent fins.

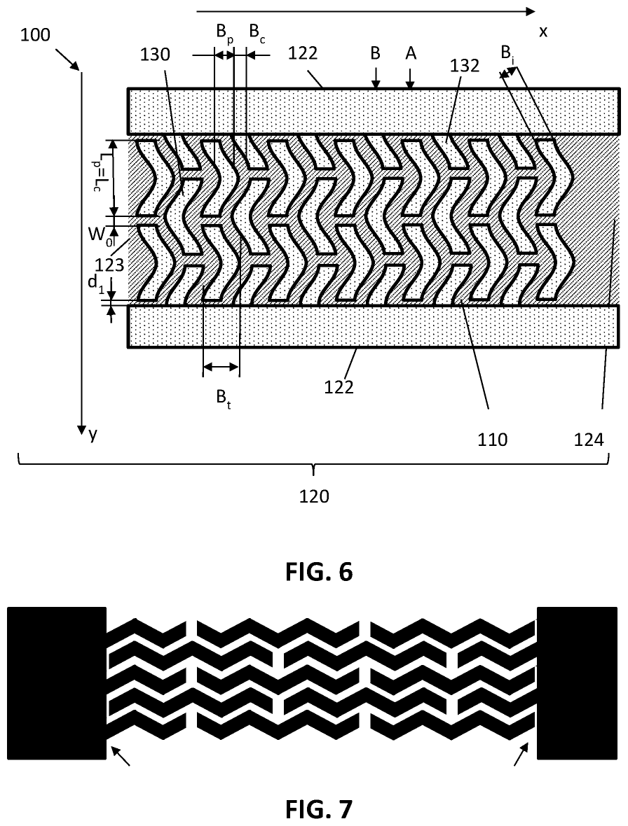

Similarly, in FIG. 6, the corners are rounded at the interface between two fins. In addition, the fins are curved such that the cross-section of the pillar and the substrate has an undulating form, and such that the width Bₚ of the flow pores between two rows of pillars is substantially the same throughout. In this example, the lateral ends of the fins are flat and parallel to the channel side wall. In the present invention, the shape obtained is also referred to as an undulating chevron structure.

In embodiments according to the present invention, the pillars 130 are microfabricated pillars.

In embodiments of the present invention, one or more additional components may also be present in a microfluidic device according to embodiments of the present invention, depending on the functionality of the microfluidic device as understood by a person skilled in the art. In some embodiments, for example, one or more distributors may be present, a detector may be present, perhaps but not necessarily integrated into one of the substrates of the microfluidic device, a further microfluidic network may be present, electrodes may be present (for example, in a microfluidic device such as a chemical reactor based on electrophoresis or an electrochemical reactor), a membrane or a filter may be present, a catalyser bed may be present, a heat exchanger may be present, a radiation source may be present, etc.

Depending on the flow opening through which a liquid fraction flows into the microfluidic device, the liquid fraction will travel along a different flow path. It is an advantage of embodiments of the present invention that the various flow paths are of equal length due to the specific arrangement of the pillars.

In a second aspect, the present invention relates to a mask for lithographically applying a structure in a substrate for the manufacture of a microfluidic device.

The mask comprises design elements for defining an ordered set of pillars 130 positioned in the channel 120 on the substrate 110. The pillars 130 have at least one pair of fins that form a chevron-shaped cross-section with the substrate.

The pillars 130 are arranged in pairs of rows. Each pair contains one row with a concave side of a pair of fins facing the average liquid flow, and another row with a convex side of a pair of fins facing the average liquid flow. Microchannels are formed between the pillars.

In embodiments of the present invention, the rows are staggered, the microchannels between pillars of successive rows having substantially the same width.

In embodiments of the present invention, the chevron form is such that a substantially constant microchannel width is obtained between two adjacent pillars of the same row.

In embodiments of the present invention, the design elements are defined in such a way that the resulting pillars touching the wall contain only one fin. This fin is preferably oriented in the direction of the liquid flow.

In embodiments of the present invention, the fins against the channel wall may, for example, form part of the first rows. In other embodiments, by contrast, they form part of the second rows.

In general, the mask according to the second aspect of the present invention may, for the lithographic application of the pillar structure, be formed in such a way that it is suitable for the application of a pillar structure according to the first aspect of the present invention.

In a third aspect, the present invention relates to a method for production of a microfluidic device. The method comprises the lithographic implementation of a channel with pillars using a mask in accordance with embodiments of the present invention. The method may include the step of lithographically transferring the pattern of a mask onto a substrate to generate substrate features and etching the substrate features to generate pillars. Other characteristics of the manufacturing process of the microfluidic device can be understood by a person skilled in the art, and are therefore not described in further detail here.

In an embodiment of the present invention, the pillars, inlet and outlet are produced by transferring the design to a silicon substrate using deep UV photolithography.

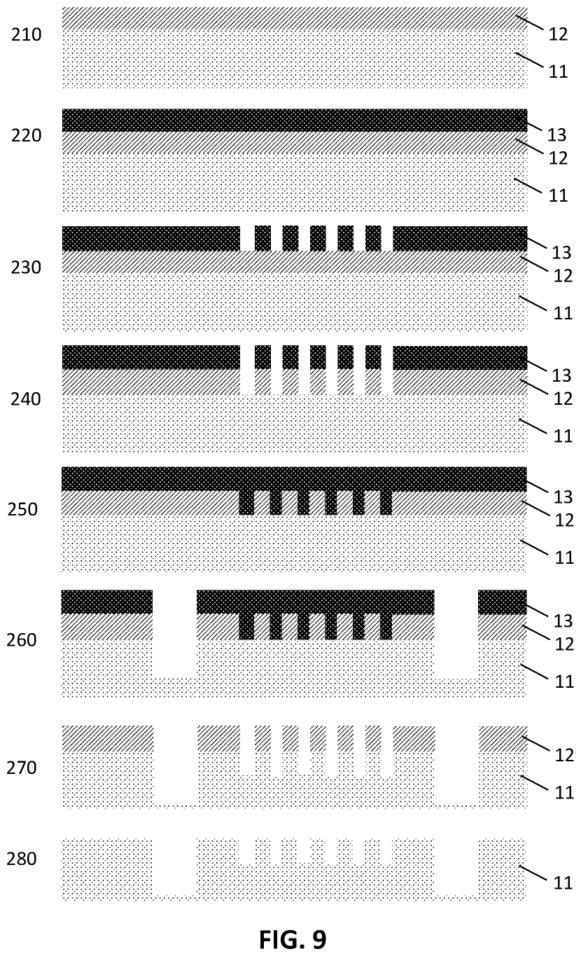

An example of this is shown in FIG. 9. For this purpose, for example, first (step 210) a layer of silicon oxide 12 is applied with a thickness of 1 μm to a silicon substrate 11. This layer acts as a hard mask for the separation bed after the inlet and outlet channels have been formed following a second exposure.

Next (step 220), a photosensitive lacquer 13 is applied to the hard mask 12.

During the first exposure, the pattern of the pillar bed is transferred into this hard mask layer by an initial dry etching step (230, 240).

A second photolithographic cycle is then carried out, which starts (step 250) with the application of a new layer of photosensitive lacquer (13), an exposure which defines the inlet and outlet channels in the lacquer, followed by a series of dry deep etching steps in order to form the inlet and outlet channels, for example, to a depth of 100 μm (step 260).

By removing the photo lacquer 13, the pattern of the pillar bed formed in the hard mask layer is released, and the pillar bed can be formed to a depth of, for example, 30 μm by a subsequent series of three steps, the inlet and outlet channels being further deepened to 130 μm (step 270).

In the final step 280, the hard mask layer 12 can be removed by a modified wet etching step. To obtain a closed reactor, the etched silicon substrate can be anchored to a borosilicate glass substrate, for example, by means of an anodic bonding step.

The various aspects can be easily combined with one another, and the combinations therefore also correspond to embodiments in accordance with the present invention.

1. A microfluidic device (100) based on a liquid flow, comprising the microfluidic device (100):
a substrate (110) having a liquid channel (120) defined by channel walls (122), the channel (120) having an inlet (123) and an outlet (124), and the channel (120) having a longitudinal axis in accordance with the average liquid flow direction of a liquid as it flows into the channel (120) from the inlet (123) to the outlet (124),
an ordered set of pillars (130) positioned in the channel (120) on the substrate (110), the individual pillars (130) comprising at least one pair of fins, the fins forming a chevron-shaped cross-section with the substrate,
and where the pillars (130) are arranged in pairs of rows, adjacent rows being laterally displaced with respect to one another by half a pillar length, the pillar length being measured perpendicular to the average liquid direction,
and where the rows are staggered so that the microchannels formed between pillars of successive rows at each position along the longest pillar side have substantially the same width.

2. A microfluidic device (100) according to claim 1, where the chevron form is such that a substantially constant microchannel width is obtained between two adjacent pillars of the same row.

3. A microfluidic device (100) according to claim 1, where the ratio of the total width Bₜ of a pillar, measured in the average liquid flow direction, and the average width Bᵢ of the pillar, measured perpendicular to the wall of a fin, is greater than 1.05.

4. A microfluidic device (100) according to claim 1, where the pillars that touch the channel walls (122) contain only half the fins of a pillar that does not touch the channel wall.

5. A microfluidic device (100) according to claim 1, where, in one row of a pair of rows, the outer pillars touch the channel walls, and where there are flow openings between the outer pillars and the channel walls for the other row of the pair of rows.

6. A microfluidic device (100) according to claim 1, where, for one row of a pair of rows, a flow opening is present between an outermost pillar and the first channel wall, and a pillar on the other side touches a second channel wall opposite the first channel wall, and where, for another row of the pair of rows, a flow opening is present between the outermost pillar and the second channel wall, and a pillar on the other side touches the first channel wall.

7. A microfluidic device (100) according to claim 1, where connections between two fins and the ends of fins are rounded.

8. A microfluidic device (100) according to claim 1, where the ratio of the height of the pillars and the width of the pillars is greater than three, where the height of the pillars is measured in a direction orthogonal to the substrate (110).

9. A microfluidic device (100) according to claim 1, where the fins of the pillars (130) have a width (Bₚ) in the direction of the longitudinal axis of the channel (120), and where the chevron shapes have a length (L_{c}) in a direction perpendicular to the longitudinal axis and parallel to the substrate, and where the individual chevron shapes have a length-width ratio of at least three.

10. A microfluidic device (100) according to claim 1, where the ends of the fins are parallel to the channel walls (122).

11. A microfluidic device (100) according to claim 1, with the microfluidic device comprising a top plate on top of the pillars (130) and the top plate being positioned opposite the substrate (110).

12. A microfluidic device (100) according to claim 1, where the smallest distance (W0) between two adjacent pillars (130) is between 0.5 times and 1.1 times the smallest distance (d1) between the channel wall (122) and an adjacent, non-contacting pillar.

13. A microfluidic device (100) according to claim 1, where a double node is a flow opening between adjacent pillars of the same row, where two microchannels arrive and two microchannels depart, where the microchannels that arrive in a double node and the microchannels that depart in a double node are symmetrical.

14. A mask for lithographical application of a structure into a substrate for the manufacture of a microfluidic device (100), comprising:
design elements for defining an ordered set of pillars (130) positioned in the channel (120) on the substrate (110), the individual pillars (130) having at least one pair of fins that form a chevron-shaped cross-section with the substrate,
and where the pillars (130) are arranged in pairs of rows, adjacent rows being laterally displaced with respect to one another by half a pillar length, the pillar length being measured perpendicular to the average liquid direction, thereby forming microchannels between the pillars,
and where the rows are staggered so that the microchannels formed between pillars of successive rows have substantially the same width.

15. A method for producing a microfluidic device, the method comprising the lithographic implementation of a channel with pillars using a mask in accordance with claim 14.
